(19) United States
(12) Patent Application Publication
(10) Pub. No.: US 2022/0080165 A1
(43) Pub. Date: Mar. 17, 2022

(54) GRIP ADJUSTMENT ASSEMBLY FOR ENT INSTRUMENT

(71) Applicant: Acclarent, Inc., Irvine, CA (US)

(72) Inventors: Jetmir Palushi, Irvine, CA (US); Hany Abdelwahed, Irvine, CA (US); Madison K. Vanosdoll, Cincinnati, OH (US); Karthick Ramyan Mohan, Irvine, CA (US)

(21) Appl. No.: 17/464,809

(22) Filed: Sep. 2, 2021

Related U.S. Application Data
(60) Provisional application No. 63/078,610, filed on Sep. 15, 2020.

(51) Int. Cl. A61M 29/00

(52) U.S. Cl. CPC A61M 29/00; A61M 2210/065; A61M 2210/0662; A61M 2210/0618

(57) ABSTRACT

An apparatus includes a guide member, a handle assembly, a gripping assembly, and a grip adjustment assembly. The handle assembly extends proximally from the guide member. The handle assembly includes a first engagement feature. The gripping assembly includes a second engagement feature. The gripping assembly is configured to selectively transition between a locked configuration and a movable configuration. In the locked configuration, the first and second engagement features are configured to engage one another to prevent relative movement of the gripping assembly relative to the handle assembly. The grip adjustment assembly includes a cam feature disposed on at least one of the handle assembly or the gripping assembly. In the movable configuration, the cam feature of the grip adjustment assembly is configured to flex the second engagement feature away from the first engagement feature allowing the gripping assembly to move relative to the handle assembly.

### PRIORITY

This application claims priority to U.S. Provisional Pat. App. No. 63/078,610, entitled “Grip Adjustment Assembly for ENT Instrument,” filed Sep. 15, 2020, the disclosure of which is incorporated by reference herein, in its entirety.

### BACKGROUND

In some instances, it may be desirable to dilate an anatomical passageway in a patient. This may include dilation of ostia of paranasal sinuses (e.g., to treat sinusitis), dilation of the larynx, dilation of the Eustachian tube, dilation of other passageways within the ear, nose, or throat, etc. A system to perform such procedures is disclosed in U.S. Pat. Pub. No. 2011/0004057, entitled “Systems and Methods for Transnasal Dilation of Passageways in the Ear, Nose or Throat,” published Jan. 6, 2011, now abandoned, the disclosure of which is incorporated by reference herein, in its entirety.

In the context of Eustachian tube dilation, a dilation catheter or other dilation instrument may be inserted into the Eustachian tube and then be inflated or otherwise expanded to thereby dilate the Eustachian tube. Methods and devices for dilating the Eustachian tube are disclosed in U.S. Pat. Pub. No. 2010/0274188, entitled “Method and System for Treating Target Tissue within the ET,” published on Oct. 28, 2010, now abandoned, the disclosure of which is incorporated by reference herein, in its entirety; and U.S. Pat. Pub. No. 2013/0274715, entitled “Method and System for Eustachian Tube Dilation,” published on Oct. 17, 2013, now abandoned, the disclosure of which is incorporated by reference herein, in its entirety.

While a variable direction view endoscope may be used to provide visualization within the anatomical passageway, it may also be desirable to provide additional visual confirmation of the proper positioning of the balloon before inflating the balloon. This may be done using an illuminating guidewire. Such a guidewire may be positioned within the target area and then illuminated, with light projecting from the distal end of the guidewire. This light may illuminate the adjacent tissue (e.g., hypodermis, subdermis, etc.) and thus be visible to the naked eye from outside the patient through transcutaneous illumination. Such an illuminating guidewire may be provided in accordance with the teachings of U.S. Pat. No. 9,155,492, entitled “Sinus Illumination Lightwire Device,” issued Oct. 13, 2015, the disclosure of which is incorporated by reference herein, in its entirety.

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.), such that the computer system may superimpose the current location of the instrument on the preoperatively obtained images. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, special instruments having sensors (e.g., electromagnetic coils that emit electromagnetic fields and/or are responsive to externally generated electromagnetic fields) are used to perform the procedure while the sensors send data to the computer indicating the current position of each surgical instrument. The computer correlates the data it receives from the sensors with the digital map that was created from the preoperative tomographic scan. The tomographic scan images are displayed on a video monitor along with an indicator (e.g., crosshairs or an illuminated dot, etc.) showing the real-time position of each surgical instrument relative to the anatomical structures shown in the scan images.

When applied to functional endoscopic sinus surgery (FESS), balloon sinuplasty, and/or other ENT procedures, the use of IGS systems allows the surgeon to achieve more precise movement and positioning of the surgical instruments than can be achieved by viewing through an endoscope alone. Examples of use of an IGS system in an ENT procedure are described in U.S. Pat. Pub. No. 2014/0364725, entitled “Systems and Methods for Performing Image Guided Procedures within the Ear, Nose, Throat and Paranasal Sinuses,” published Dec. 11, 2014, now abandoned; and U.S. Pat. No. 10,561,370, entitled “Apparatus to Secure Field Generating Device to Chair,” issued Feb. 18, 2020, the disclosures of which are incorporated by reference herein, in its entirety.

While several systems and methods have been made and used to position a balloon of a dilation catheter in an anatomical passageway, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1A depicts a front perspective view of an exemplary dilation instrument that includes a guide shaft assembly, a handle assembly, and a gripping assembly, with a guidewire and a dilation catheter each in respective proximal positions;

FIG. 1B depicts a front perspective view of the instrument of FIG. 1A, with the guidewire in a distal position and the dilation catheter in the proximal position;

FIG. 1C depicts a front perspective view of the instrument of FIG. 1A, with the guidewire and the dilation catheter each in respective distal positions, and with a dilator of the dilation catheter in a non-expanded state;

FIG. 1D depicts a front perspective view of the instrument of FIG. 1A, with the guidewire and the dilation catheter each in respective distal positions, and with a dilator of the dilation catheter in an expanded state;

FIG. 2 depicts a partially exploded front perspective view of a body of the handle assembly and the gripping assembly of FIG. 1A and an exemplary grip adjustment assembly, with the body of the handle assembly and the gripping assembly including engagement features;

FIG. 3 depicts an enlarged partially exploded rear perspective view of the body of the handle assembly, the gripping assembly, and the grip adjustment assembly of FIG. 2;

FIG. 4A depicts a side elevational view of the body of the handle assembly and the gripping assembly of FIG. 3 in a locked configuration exposing the grip adjustment assembly, with the gripping assembly in a first longitudinal position;

FIG. 4B depicts a side elevational view of the body of the handle assembly and the gripping assembly of FIG. 4A, but in a movable configuration where the body of the handle assembly and the gripping assembly are squeezed together using the grip adjustment assembly with the gripping assembly in the first longitudinal position;

FIG. 4C depicts a side elevational view of the body of the handle assembly and the gripping assembly of FIG. 4B in the movable configuration where the body of the handle assembly and the gripping assembly are squeezed together using the grip adjustment assembly while the gripping assembly is moved relative to the handle assembly to a second longitudinal position;

FIG. 4D depicts a side elevational view of the body of the handle assembly and the gripping assembly of FIG. 4C in the locked configuration exposing the grip adjustment assembly, with the gripping assembly at the second longitudinal position;

FIG. 5A depicts a cross-sectional view of the body of the handle assembly and the gripping assembly of FIG. 4A taken along line 5A-5A of FIG. 4A in the locked configuration exposing the grip adjustment assembly;

FIG. 5B depicts a cross-sectional view of the body of the handle assembly and the gripping assembly of FIG. 4B taken along line 5B-5B of FIG. 4B in the movable configuration using the grip adjustment assembly;

FIG. 6A depicts a cross-sectional view of the body of the handle assembly and the body of the gripping assembly of FIG. 5A taken along line 6A-6A of FIG. 5A, with the first and second engagement features engaged in the locked configuration;

FIG. 6B depicts a cross-sectional view of the body of the handle assembly and the gripping assembly of FIG. 5B taken along line 6B-6B of FIG. 5B, with the engagement features disengaged in the movable configuration; and

FIG. 7 depicts a diagrammatic view of an exemplary method of using the instrument of FIG. 1A.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. For example, while various. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top” and “bottom” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Exemplary Dilation Instrument
A. Overview

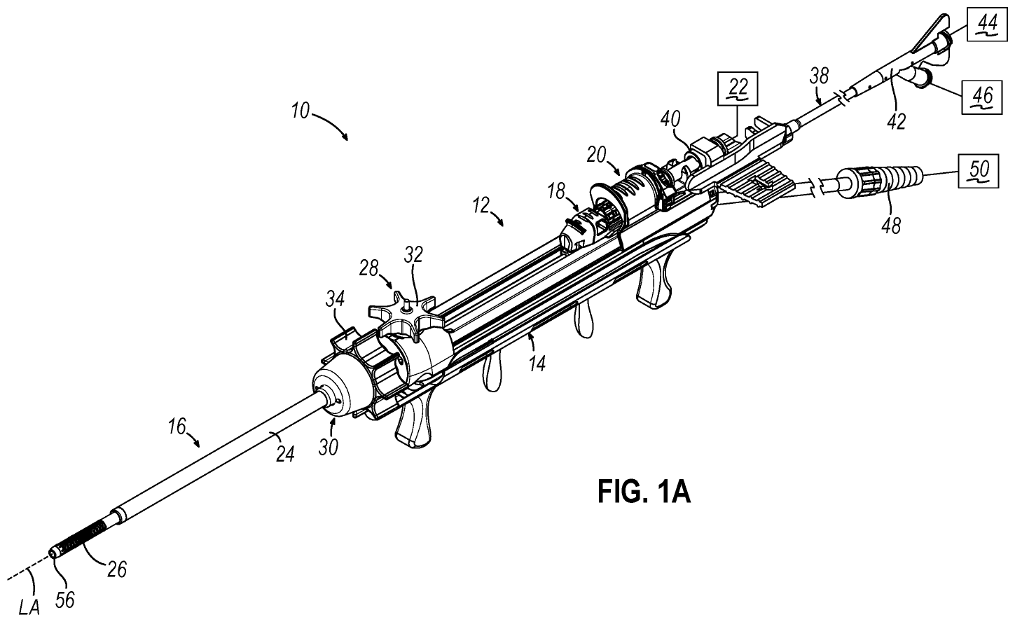

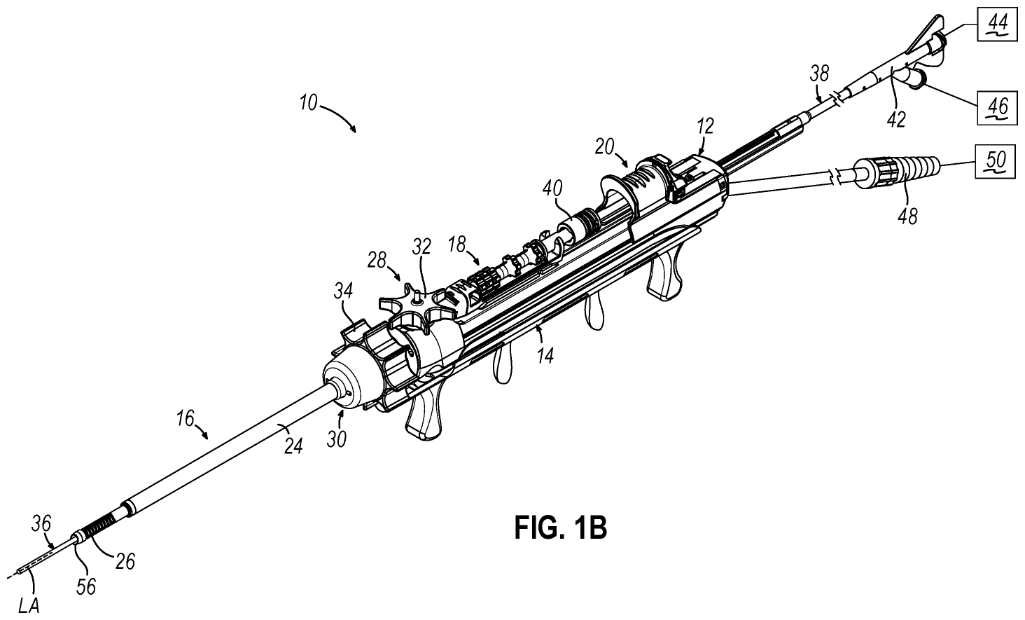

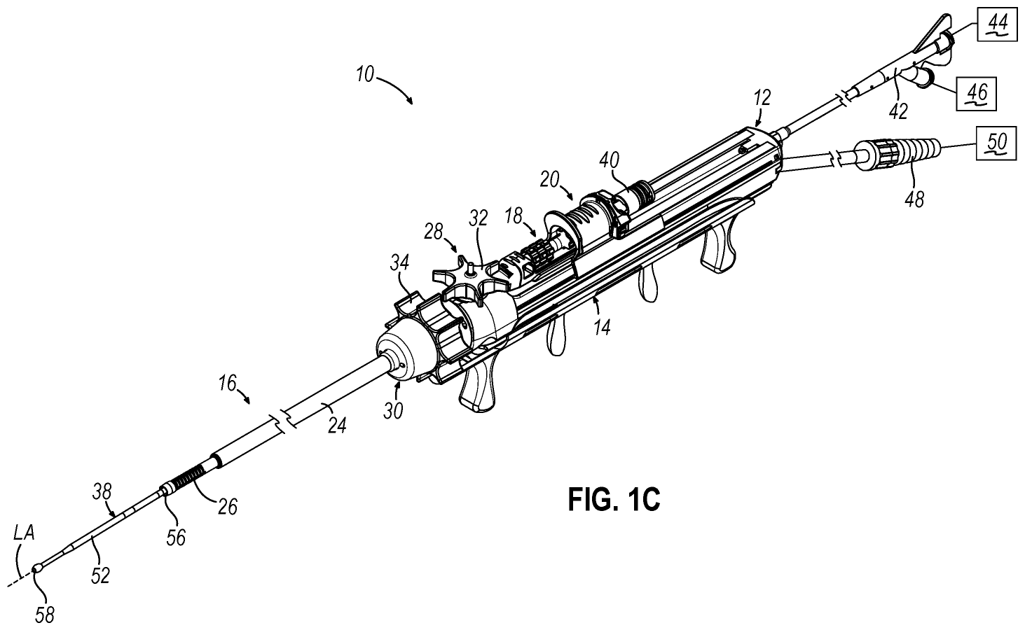

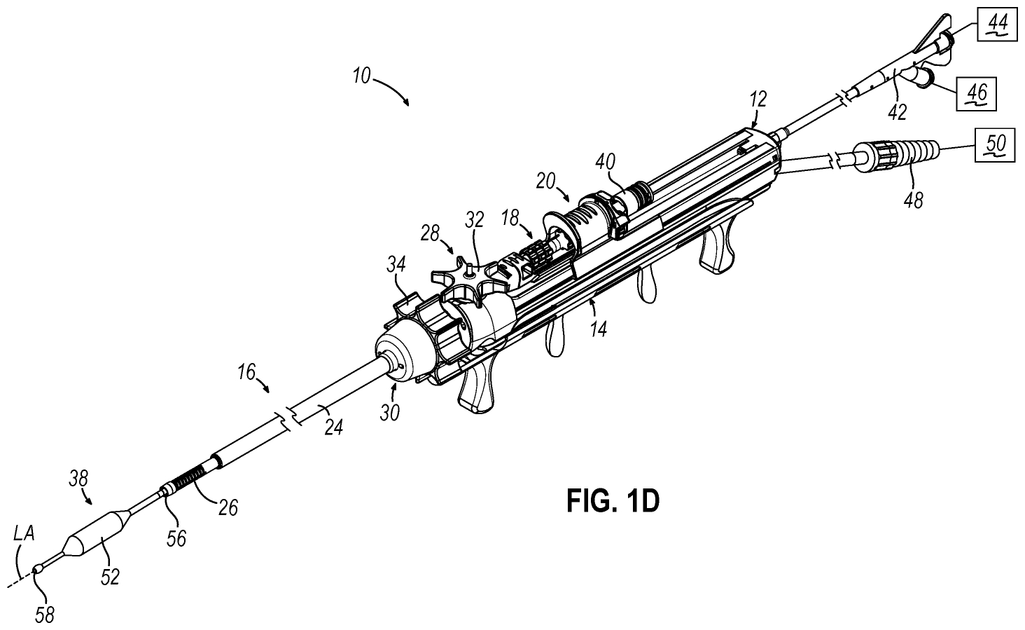

FIGS. 1A-1D show an exemplary dilation instrument (10) that may be used to dilate the ostium of a paranasal sinus, to dilate another passageway associated with drainage of a paranasal sinus, to dilate a Eustachian tube, or to dilate some other anatomical passageway (e.g., within the ear, nose, or throat, etc.). As will be described in greater detail below, dilation instrument (10) of the present example provides adjustability that enables the operator to use dilation instrument (10) in different scenarios, without requiring the operator to switch between different instruments. For instance, dilation instrument (10) may be used to dilate various different anatomical passageways (e.g., frontal sinus ostium, frontal recess, maxillary sinus ostium, sphenoid sinus ostium, ethmoid sinus ostium, Eustachian tube, etc.) by making simple adjustments to structural features of the instrument.

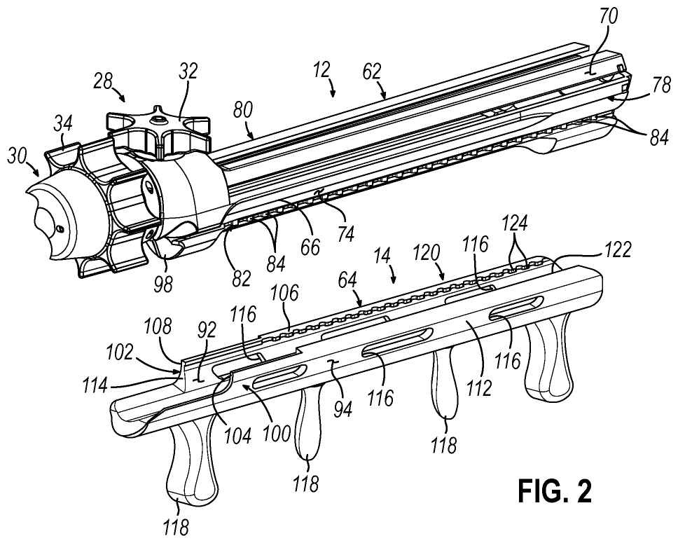

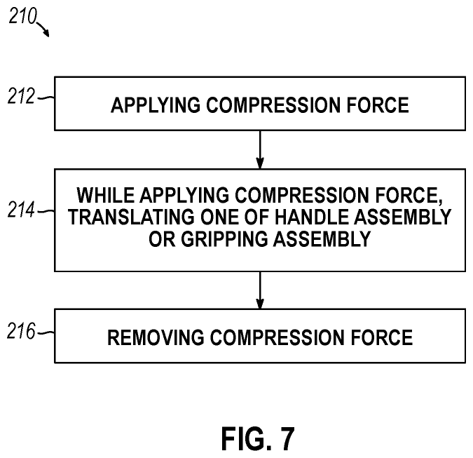

Dilation instrument (10) may include a handle assembly (12), a gripping assembly (14), a guide member (e.g., guide shaft assembly (16)), a guidewire actuation assembly (18), a dilation catheter actuation assembly (20), and a guidewire module (22). Handle assembly (12) is sized and configured to be grasped and operated by a single hand of an operator. The operator may selectively operate guidewire actuation assembly (18) and dilation catheter actuation assembly (20) with the same single hand that grasps handle assembly (12). Gripping assembly (14) is selectively coupled with handle assembly (12) as described in detail below with reference to FIGS. 2-7.

The guide member is operable to guide an instrument into a head of a patient. As shown, guide shaft assembly (16) extends distally from handle assembly (12). Guide shaft assembly (16) includes a rigid shaft member (24), a flexible shaft member (26), and actuators, such as a deflection control assembly (28) and a shaft rotation assembly (30). As shown in FIGS. 1A-1D, handle assembly (12), gripping assembly (14), and guide shaft assembly (16) extends along a longitudinal axis (LA). Rotation of knob (32) of deflection control assembly (28) in a first direction causes a push-pull wire (not shown) to move proximally causing a portion of flexible shaft member (26) to rotate proximally bend to a deflected configuration (not shown). Rotation of knob (32) in a second direction causes flexible shaft member (26) to bend back to a straight configuration. Rotation of shaft rotation assembly (30) causes guide shaft assembly (16) to rotate about longitudinal axis (LA). While knob (32) of deflection control assembly (28) is shown as being disposed perpendicular to longitudinal axis (LA) and knob (34) of shaft rotation assembly (30) is shown as being coaxial with longitudinal axis (LA), other variations are also envisioned.

Guide shaft assembly (16) defines a lumen configured to receive at least one of a guidewire (36) or a dilation catheter (38) therethrough. Guidewire actuation assembly (18) and dilation catheter actuation assembly (20) are slidably coupled with handle assembly (12). Guidewire actuation assembly (18) is configured to translate guidewire (36) relative to handle assembly (12) and to rotate guidewire (36) about longitudinal axis (LA). A connector (40) couples guidewire module (22) with guidewire (36). A connector (42) couples an inflation fluid source (44) and an irrigation fluid source (46) with dilation catheter (38). A suction port (48) and a conduit (not shown), which spans through handle assembly (12), couples suction source (50) with guide shaft assembly (16). Dilation catheter (38) is slidable relative to guide shaft assembly (16). Dilation catheter (38) includes an expandable dilator (52) (e.g., an expandable balloon). While in the present example, dilation catheter (38) is coaxially disposed within guide shaft assembly (16), and guidewire (36) is coaxially disposed within dilation catheter (38), other variations are also envisioned.

As shown in the transition from FIG. 1A to FIG. 1B, the operator may distally advance guidewire actuation assembly (18) along handle assembly (12) to distally advance guidewire (36), such that a distal end (54) of guidewire (36) is positioned distal to distal end (56) of guide shaft assembly (16). As shown in the transition from FIG. 1B to FIG. 1C, the operator may distally advance dilation catheter actuation assembly (20) along handle assembly (12) to distally advance dilation catheter (38), such that a distal tip (58) of dilation catheter (38) is positioned distal to a distal end (56) of guide shaft assembly (16). With dilation catheter (38) advanced to a desired position, the operator may then inflate dilator (52) of dilation catheter (38) to achieve an expanded state as shown in FIG. 1D, to thereby dilate an anatomical passageway in which dilator (52) is positioned.

In addition to the teachings herein, dilation instrument (10) may be configured and operable in accordance with at least some of the teachings of U.S. patent application Ser. No. 17/239,761, entitled Shaft Deflection Control Assembly for ENT Guide Instrument,” filed Apr. 26, 2021, the disclosure of which is incorporated by reference herein, in its entirety; U.S. Pat. No. 10,874,839, entitled “Adjustable Instrument for Dilation of Anatomical Passageway,” issued Dec. 29, 2020, the disclosure of which is incorporated by reference herein, in its entirety; and/or U.S. Pat. No. 11,027,105, entitled “Adjustable Instrument for Dilation of Anatomical Passageway,” issued Jun. 8, 2021, the disclosure of which is incorporated by reference herein, in its entirety. Other variations of the features and functionalities described herein will be apparent to those skilled in the art in view of the teachings herein.
B. Exemplary Grip Adjustment Assembly

In some prior versions, to adjust the longitudinal position of gripping assembly (14) relative to handle assembly (12), the user may have needed to apply a high shear force to forcibly decouple gripping assembly (14) from handle assembly (12) or to otherwise adjust the longitudinal position of gripping assembly (14) relative to handle assembly (12). This high shear force may shear or damage the structure that couples gripping assembly (14) with handle assembly (12). In addition, or in the alternative, the high shear force may be difficult for some users to achieve. While the high shear force may make adjusting the position of gripping assembly (14) relative to handle assembly (12) more difficult, the high shear force may ensure that gripping assembly (14) does not unintentionally slip relative to handle assembly (12) during the surgical procedure. In some prior versions, to adjust the longitudinal position of gripping assembly (14) relative to handle assembly (12), gripping assembly (14) may have been completely separated from handle assembly (12) then reunited with handle assembly (12) once the components are in the desired position. As a result, it may be desirable to allow the user to reduce the adjustment force when gripping assembly (14) is moved relative to handle assembly (12) without completely separating gripping assembly (14) from handle assembly (12), and without reducing the engagement force coupling handle assembly (12) with gripping assembly (14).

As will be described in greater detail below with reference to FIGS. 2-7, dilation instrument (10) using an exemplary grip adjustment assembly (60) allows the user to selectively adjust gripping assembly (14) relative to handle assembly (12) to accommodate different user gripping techniques and/or different user hand sizes without completely separating gripping assembly (14) from handle assembly (12); and without requiring the user to impart substantial shear forces on gripping assembly (14) and handle assembly (12).
1. Exemplary Cam Feature of Grip Adjustment Assembly

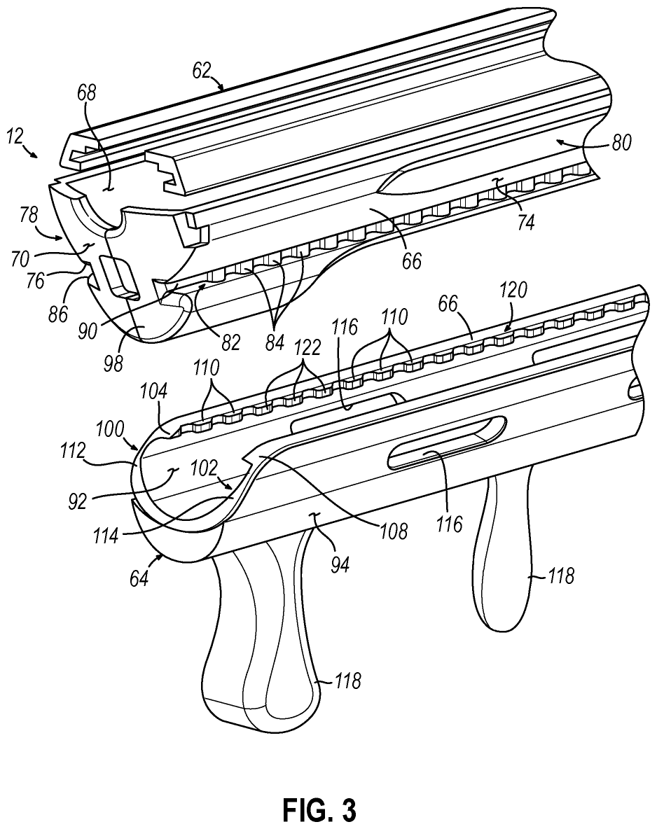

FIGS. 2-6 show handle assembly (12) and gripping assembly (14) of FIGS. 1A-1D as well as grip adjustment assembly (60) that allows for adjustment of gripping assembly (14) relative to handle assembly (12) to accommodate different user gripping techniques and/or different user hand sizes. In other words, grip adjustment assembly (60) may provide for reliable and secure readjustment of gripping assembly (14) to accommodate different user profile handling. FIGS. 2 and 3 show partially exploded perspective views of a body (62) of handle assembly (12) and a body (64) of gripping assembly (14) of FIG. 1A, where handle assembly (12) and gripping assembly (14) may be selectively coupled together using grip adjustment assembly (60). As will be described in greater detail below, grip adjustment assembly (60) includes a cam feature (66) disposed on at least one of handle assembly (12) or gripping assembly (14).

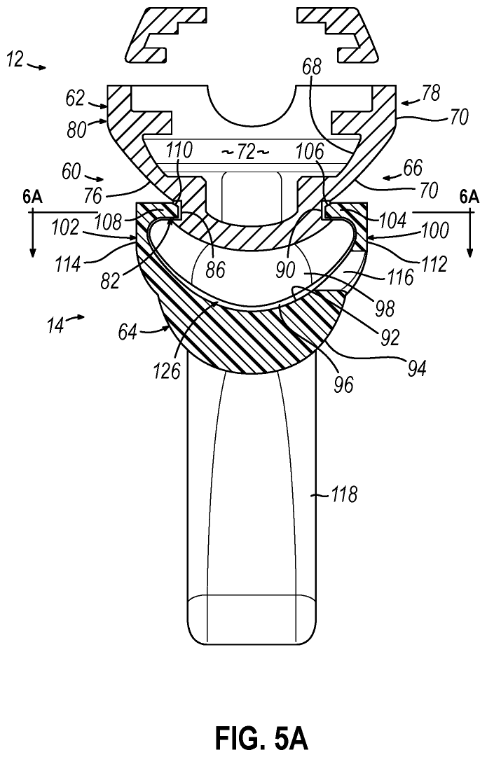

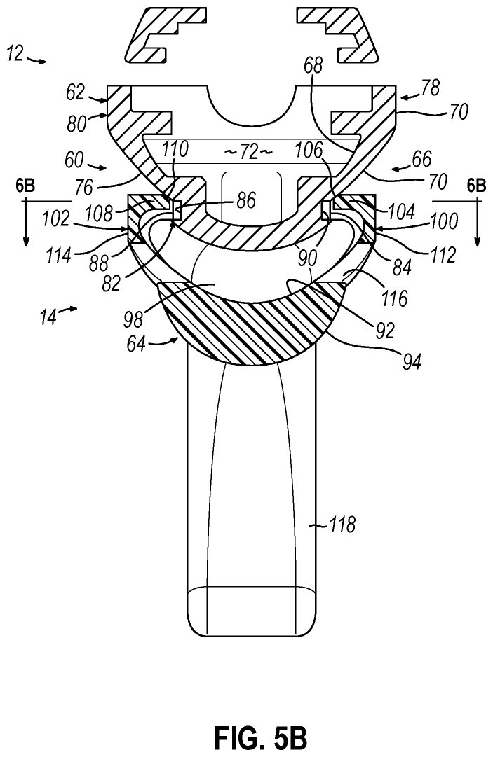

Handle assembly (12) extends proximally from guide shaft assembly (16) along longitudinal axis (LA). Body (62) of handle assembly (12) includes inner and outer surfaces (68, 70). Inner surface (68) defines a cavity (72) configured to slidably receive guidewire actuation assembly (18) and dilation catheter actuation assembly (20) within cavity (72). As shown, cam feature (66) of grip adjustment assembly (60) includes left and right cam surfaces (74, 76) disposed on outer surface (70) of handle assembly (12). Left cam surface (74) is disposed on a left lateral side (78) of body (62). Similarly, right cam surface (76) is disposed on a right lateral side (80) of body (62), opposite from left cam surface (74). Left and right cam surfaces (74, 76) are shown as outwardly projecting surfaces. Left and right cam surfaces (74, 76) act as ramps as shown in the transition from FIG. 5A to FIG. 5B to selectively couple handle assembly (12) with gripping assembly (14). Handle assembly (12) may be formed using first and second portions that are coupled together.

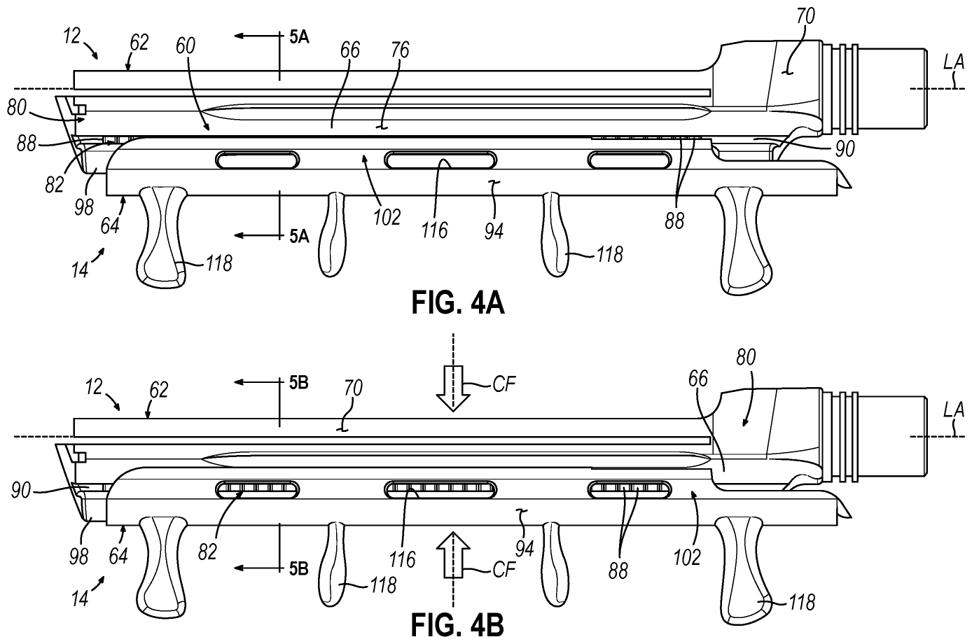

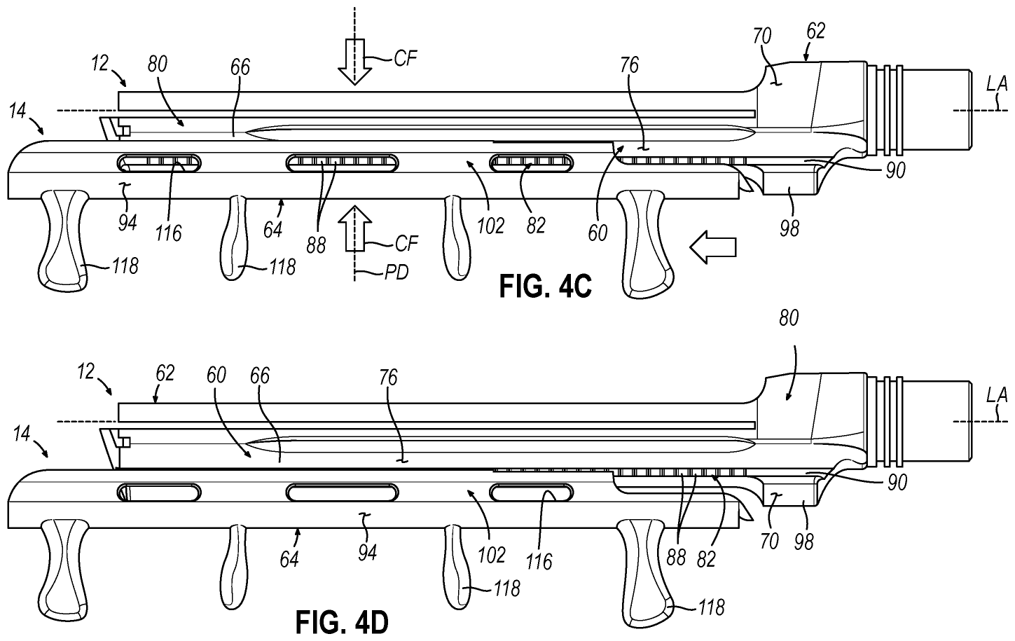

Gripping assembly (14) is selectively coupled with handle assembly (12) and is configured to transition between a locked configuration (see FIGS. 4A and 4D) and a movable configuration (see FIGS. 4B-4C). Body (64) of gripping assembly (14) includes inner and outer surfaces (92, 94). Inner surface (92) defines a cavity (96) configured to receive a lower arcuate portion (98) of handle assembly (12) therein. Body (64) of gripping assembly (14) includes left and right arms (100, 102) that collectively define lateral sides of cavity (96). Left arm (100) includes a left inwardly facing projection (104). Left inwardly facing projection (104) includes a left cam surface (106), which is shown as a planar notch in FIG. 5A. Similarly, right arm (102) includes a right inwardly facing projection (108). Right inwardly facing projection (108) includes a right cam surface (110), which is shown as an oppositely facing planar notch in FIG. 5A. As shown in FIG. 5B, left and right intermediate portions (112, 114) of left and right arms (100, 102) may flex outwardly due to force exerted by left and right cam surfaces (74, 76) of handle assembly (12) in the movable configuration. Left and right intermediate portions (112, 114) include a plurality of cavities (116) extending therethrough.

Gripping assembly (14) includes a plurality of gripping handles (118) configured to be gripped by a user. As shown, gripping handles (118) are shaped as knobs that extend generally perpendicular to longitudinal axis (LA) of handle assembly (12); however, a variety of other suitable gripping handles are also envisioned. Gripping assembly (14) includes an engagement feature (120) that includes left and right engagement teeth (122, 124). Particularly, left inwardly facing projection (104) of left arm (100) includes left engagement teeth (122). Similarly, right inwardly facing projection (108) of right arm (102) includes right engagement teeth (124). Gripping assembly (14) may be formed of an elastomeric material, which may be partially flexible. Gripping assembly (14) may be integrally formed together as a unitary piece.
2. Exemplary Method of Using Dilation Instrument

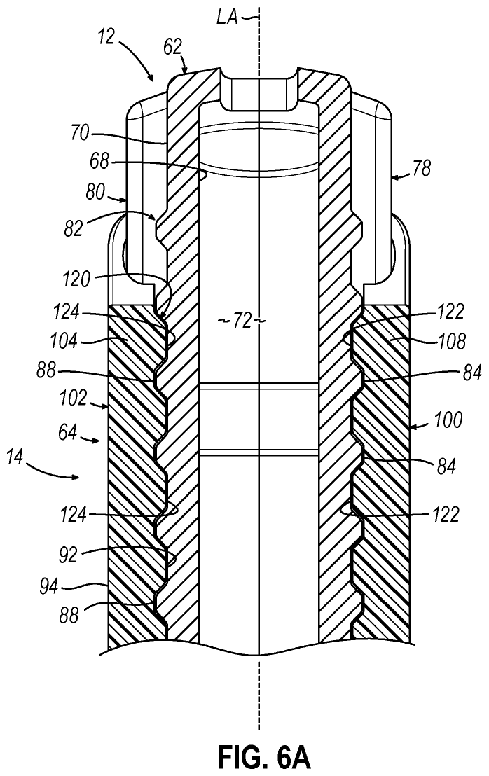

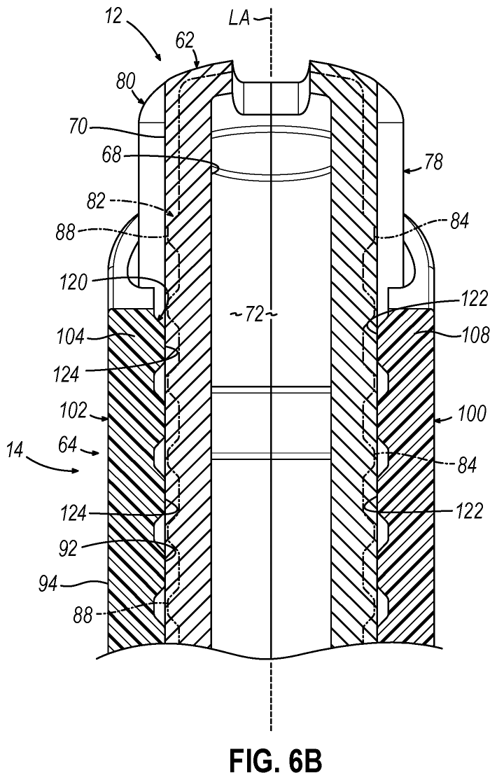

An exemplary method (210) of using dilation instrument (10) is described below with reference to FIGS. 4A-7. Dilation instrument (10) is movable between a locked configuration and a movable configuration using cam feature (66) of grip adjustment assembly (60). FIGS. 4A-4D show an exemplary adjustment of gripping assembly (14) relative to handle assembly (12) using grip adjustment assembly (60), with FIGS. 5A and 6A providing additional clarity regarding the locked configuration and FIGS. 5B and 6B providing additional clarity regarding the movable configuration. While FIGS. 4A-4B show gripping assembly (14) disposed at a first longitudinal position relative to handle assembly (12) and FIGS. 4C-4D show gripping assembly (14) disposed at a second longitudinal position relative to handle assembly (12), a variety of other suitable positions (e.g., intermediate positions) are also envisioned.

While FIGS. 4A, 5A, and 6A show handle assembly (12) and gripping assembly (14) in the locked configuration, handle assembly (12) and gripping assembly (14) may be completely separate from one another. Particularly, FIG. 4A shows a side elevational view of body (62) of handle assembly (12) and gripping assembly (14) of FIG. 3 in the locked configuration exposing grip adjustment assembly (60), with gripping assembly (14) disposed at a first longitudinal position relative to handle assembly (12). In the locked configuration, engagement feature (82) of handle assembly (12) is at least partially engaged with engagement feature (120) of gripping assembly (14) to prevent relative movement (e.g., due to slippage) of gripping assembly (14) relative to handle assembly (12). As shown, preventing relative movement of gripping assembly (14) relative to handle assembly (12) may include prevent relative translation of gripping assembly (14) relative to handle assembly (12).

FIG. 5A shows a cross-sectional view of body (62) of handle assembly (12) and gripping assembly (14) of FIG. 4A taken along line 5A-5A of FIG. 4A in the locked configuration exposing grip adjustment assembly (60). As shown, left engagement teeth (122) of engagement feature (120) are disposed in left recess (86) of left lateral side (78). Similarly, right engagement teeth (124) of engagement feature (120) are disposed in right recess (90) of right lateral side (80). Additionally, left and right cam surfaces (106, 110) of left and right arms (100, 102) are separated a distance from left and right cam surfaces (74, 76) of handle assembly (12) in the locked configuration. FIG. 5A shows a gap (126) disposed between lower arcuate portion (98) of handle assembly (12) and inner surface (92) of gripping assembly (14) prior to handle assembly (12) and gripping assembly (14) being compressed together.

FIG. 6A shows a cross-sectional view of body (62) of handle assembly (12) and gripping assembly (14) of FIG. 5A taken along line 6A-6A of FIG. 5A, with engagement features (82, 120) engaged in the locked configuration. It is envisioned that the locked configuration may include full engagement of engagement feature (120) with engagement feature (82) or partial engagement of engagement feature (120) with engagement feature (82). While full engagement of engagement feature (120 with engagement feature (82) is shown in FIG. 6A, partial engagement of engagement feature (120) with engagement feature (82) may be sufficient to reduce resistance. For example, in the locked configuration, at least one tooth of left and right engagement teeth (122, 124) of gripping assembly (14) is in locking engagement with least one tooth of left and right engagement teeth (84, 88) of handle assembly (12). As shown in FIG. 6A, left engagement teeth (122) of engagement feature (120) are engaged with left engagement teeth (84) of engagement feature (82). Similarly, right engagement teeth (124) of engagement feature (120) are engaged with right engagement teeth (88) of engagement feature (82) in the locked configuration.

At step (212), method (210) may include applying a compression force (CF) to force a cam surface of one of handle assembly (12) or gripping assembly (14) against the other of handle assembly (12) or gripping assembly (14) to allow for relative movement (e.g., translation) between handle assembly (12) and gripping assembly (14). FIG. 4B shows a side elevational view of body (62) of handle assembly (12) and gripping assembly (14) of FIG. 4A, but in a movable configuration where body (62) of handle assembly (12) and gripping assembly (14) are squeezed together using grip adjustment assembly (60), with gripping assembly (14) in the first longitudinal position.

FIG. 5B shows a cross-sectional view of body (62) of handle assembly (12) and gripping assembly (14) of FIG. 4B taken along line 5B-5B of FIG. 4B in the movable configuration using grip adjustment assembly (60). As shown, engagement feature (82) of handle assembly (12) is disengaged from engagement feature (120) of gripping assembly (14). In the movable configuration, cam feature (66) of grip adjustment assembly (60) is configured to flex left and right arms (100, 102) outwardly from longitudinal axis (LA), such that engagement feature (118) of gripping assembly (14) moves away from engagement feature (82) of handle assembly (12) allowing gripping assembly (14) to move (e.g., translate) relative to handle assembly (12). Cam feature (66) includes left and right cam surfaces (74, 76) of handle assembly (12) and left and right cam surfaces (106, 110) of gripping assembly (14). In the movable configuration, left and right cam surfaces (106, 110) of gripping assembly (14) are configured to contact left and right cam surfaces (74, 76) of handle assembly (12) to flex engagement feature (120) away from engagement feature (82). As previously described, left and right cam surfaces (74, 76) of handle assembly (12) act as ramps to contact left and right cam surfaces (106, 110) of left and right arms (100, 102) of gripping assembly (14).

Particularly, left cam surface (106) of left arm (100) of gripping assembly (14) is configured to contact and move along left cam surface (74) of handle assembly (12) in the transition from FIG. 5A to FIG. 5B. Similarly, right cam surface (110) of right arm (102) of gripping assembly (14) is configured to contact and move along right cam surface (76) of handle assembly (12) in the transition from FIG. 5A to FIG. 5B. This also causes left and right intermediate portions (112, 114) of left and right arms (100, 102) to flex outwardly as shown in FIG. 5B. Gripping assembly (14) may made of a semi-elastic material allowing left and right arms (100, 102) to flex onto left and right cam surfaces (74, 76) of handle assembly (12). FIG. 5B shows inner surface (92) of gripping assembly (14) contacting lower arcuate portion (98) of handle assembly (12) reducing or eliminating gap (126) shown in FIG. 5A. Compression force (CF) may be applied in a generally perpendicular direction (PD) to longitudinal axis (LA). This generally perpendicular direction (PD) is shown as being generally vertical in FIGS. 4B-4C. Compression force (CF) may be applied using a single hand or multiple hands.

FIG. 6B shows a cross-sectional view of body (62) of handle assembly (12) and gripping assembly (14) of FIG. 5B taken along line 6B-6B of FIG. 5B, with engagement features (82, 120) disengaged in the movable configuration. As shown, left engagement teeth (122) of engagement feature (120) are separated a distance from left engagement teeth (84) of engagement feature (82). Similarly, right engagement teeth (124) of engagement feature (120) are separated a distance from right engagement teeth (88) of engagement feature (82) in the movable configuration.

At step (214), method (210) may also include while applying compression force (CF), translating one of handle assembly (12) or gripping assembly (14) relative to other of handle assembly (12) or gripping assembly (14). FIG. 4C shows a side elevational view of body (62) of handle assembly (12) and gripping assembly (14) of FIG. 4B in the movable configuration, where body (62) of handle assembly (12) and gripping assembly (14) are squeezed together using grip adjustment assembly (60) while gripping assembly (14) is moved relative to handle assembly (12) to a second longitudinal position. For example, once left engagement teeth (84, 122) and right engagement teeth (88, 124) are disengaged in the movable configuration, the user may move gripping assembly (14) to the desired position, while under compression. As shown, compression force (CF) may applied in a direction perpendicular to longitudinal axis (LA). While not shown with reference to FIG. 4C, the relationship between left cam surfaces (74, 106) and right cam surfaces (76, 110) of handle assembly (12) and gripping assembly (14) may be similar to FIG. 5B. Similarly, the relationship between left engagement teeth (84, 122) and right engagement teeth (88, 124) of handle assembly (12) and gripping assembly (14) may be similar to FIG. 6B.

At step (216), method (210) may also include removing compression force (CF) to prevent relative movement (e.g., translation) of handle assembly (12) and gripping assembly (14). Once the desired relative longitudinal position is achieved, the user may remove compression force (CF) (e.g., no longer squeezing handle assembly (12) and gripping assembly (14)). This allows left and right intermediate portions (112, 114) to contract and engage left and right engagement teeth (122, 124) of gripping assembly (14) with left and right engagement teeth (84, 88) of handle assembly (12). FIG. 4D shows a side elevational view of body (62) of handle assembly (12) and gripping assembly (14) of FIG. 4C in the locked configuration exposing grip adjustment assembly (60), with gripping assembly (14) at the second longitudinal position. Removing the compression force (CF) automatically engages engagement feature (82) of handle assembly (12) with engagement feature (120) of gripping assembly (14).

As shown in the transition from FIG. 4C to FIG. 4D, removing compression force (CF) causes left and right cam surfaces (106, 110) of gripping assembly (14) to move downward along left and right cam surfaces (74, 76) of handle assembly (12) into left and right recesses (86, 90) of handle assembly (12). This engages left engagement teeth (122) of gripping assembly (14) with left engagement teeth (84) of handle assembly (12) and right engagement teeth (124) of gripping assembly (14) with right engagement teeth (88) of handle assembly (12). In other words, gripping assembly (14) once no longer being actively squeezed into handle assembly (12), moves back to the locked configuration where engagement features (82, 120) are engaged to prevent relative slippage. While not shown with reference to FIG. 4D, the relationship between left cam surfaces (74, 106) and right cam surfaces (76, 110) of handle assembly (12) and gripping assembly (14) may be similar to FIG. 5A. Similarly, the relationship between the relationship between left engagement teeth (84, 122) and right engagement teeth (88, 124) of handle assembly (12) and gripping assembly (14) may be similar to FIG. 6A.

II. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

An apparatus comprising: (a) a guide member operable to guide an instrument into a head of a patient; (b) a handle assembly extending proximally from the guide member, wherein the handle assembly includes a first engagement feature; (c) a gripping assembly that includes a second engagement feature, wherein the gripping assembly is configured to selectively transition between a locked configuration and a movable configuration, wherein in the locked configuration the first and second engagement features are configured to engage one another to prevent relative movement of the gripping assembly relative to the handle assembly; and (d) a grip adjustment assembly that includes a cam feature disposed on at least one of the handle assembly or the gripping assembly, wherein in the movable configuration the cam feature of the grip adjustment assembly is configured to flex the second engagement feature away from the first engagement feature allowing the gripping assembly to move relative to the handle assembly.

### EXAMPLE 2

The apparatus of Example 1, wherein the handle assembly includes an outer surface, wherein the cam feature is disposed on the outer surface.

### EXAMPLE 3

The apparatus of any one or more of Examples 1 through 2, wherein in the movable configuration the cam feature is configured to contact the gripping assembly to flex the second engagement feature away from the first engagement feature allowing the gripping assembly to move relative to the handle assembly.

### EXAMPLE 4

The apparatus of any one or more of Examples 1 through 3, wherein in the locked configuration the first and second engagement features are configured to engage one another to prevent translation of the gripping assembly relative to the handle assembly, wherein in the movable configuration the cam feature is configured to contact the gripping assembly to flex the second engagement feature away from the first engagement feature allowing the gripping assembly to translate relative to the handle assembly.

### EXAMPLE 5

The apparatus of any one or more of Examples 1 through 4, wherein one of the handle assembly or the gripping assembly includes an outwardly projecting surface, wherein the cam feature is disposed on outwardly projecting surface

### EXAMPLE 6

The apparatus of any one or more of Examples 1 through 5, wherein the cam feature includes first and second cam surfaces disposed on opposing first and second outwardly projecting surfaces of the handle assembly.

### EXAMPLE 7

The apparatus of Example 6, wherein of the gripping assembly includes first and second arms that are configured to contact the first and second cam surfaces in the movable configuration.

### EXAMPLE 8

The apparatus of Example 7, wherein the first and second arms are configured to be separated a distance from the first and second cam surfaces in the locked configuration.

### EXAMPLE 9

The apparatus of any one or more of Examples 7 through 8, wherein the first and second arms include first and second inwardly facing projections, wherein the first and second inwardly facing projections are configured to contact the first and second cam surfaces in the movable configuration.

### EXAMPLE 10

The apparatus of any one or more of Examples 7 through 9, wherein the first and second arms are configured to flex outwardly due to force exerted by the first and second cam surfaces in the movable configuration.

### EXAMPLE 11

The apparatus of any one or more of Examples 7 through 10, wherein the first engagement feature includes a first plurality of engagement teeth, wherein the second engagement feature includes a second plurality of engagement teeth disposed on the first arm, wherein the plurality of engagement teeth are configured to engage the first plurality of engagement teeth in the locked configuration.

### EXAMPLE 12

The apparatus of any one or more of Example 11, wherein the first engagement feature includes a third plurality of engagement teeth, wherein the second engagement feature includes a fourth plurality of engagement teeth disposed on the second arm, wherein the fourth plurality of engagement teeth are configured to engage the third plurality of engagement teeth in the locked configuration.

### EXAMPLE 13

The apparatus of any one or more of Examples 11 through 12, wherein the handle assembly includes in first and second recessed portions, wherein the first and third pluralities of engagement teeth are disposed in first and second recessed portions.

### EXAMPLE 14

The apparatus of any one or more of Examples 1 through 13, wherein the first engagement feature includes a first plurality of engagement teeth, wherein the second engagement feature includes a second plurality of engagement teeth, wherein in the locked configuration at least one tooth of the first plurality of engagement teeth is configured to be in locking engagement with least one tooth of the first plurality of engagement teeth.

### EXAMPLE 15

The apparatus of any one or more of Examples 1 through 14, wherein the gripping assembly includes a plurality of gripping handles.

### EXAMPLE 16

The apparatus of any one or more of Examples 1 through 15, wherein the handle assembly includes a first body, wherein the first body includes the first engagement feature and the cam feature, wherein the gripping assembly includes a second body, wherein the second body includes the second engagement feature.

### EXAMPLE 17

The apparatus of any one or more of Examples 1 through 16, wherein the gripping assembly is formed of an elastomeric material.

### EXAMPLE 18

The apparatus of any one or more of Examples 1 through 17, further comprising a dilation catheter, the guide member being operable to guide the dilation catheter into the head of the patient, wherein the dilation catheter is slidable relative to the guide member, wherein the dilation catheter comprises an expandable dilator.

### EXAMPLE 19

The apparatus of Example 18, further comprising a guidewire, wherein the guidewire is slidably disposed in the dilation catheter.

### EXAMPLE 20

The apparatus of Example 19, further comprising a guidewire actuation assembly, wherein the guidewire actuation assembly is operable to translate the guidewire relative to the handle assembly, wherein the guidewire actuation assembly is further operable to rotate the guidewire about a longitudinal axis.

### EXAMPLE 21

The apparatus of any one or more of Examples 1 through 20, further comprising a shaft rotation assembly, wherein the shaft rotation assembly is operable to rotate the guide member about the longitudinal axis.

### EXAMPLE 22

An apparatus comprising: (a) a guide member, the guide member being operable to guide an instrument into a head of a patient; (b) a handle assembly extending proximally from the guide member, the handle assembly comprising: (i) a first engagement feature, and (ii) a cam feature disposed on an outer surface of the handle assembly; and (c) a gripping assembly that includes a second engagement feature, wherein gripping assembly is configured to selectively transition between a locked configuration and a movable configuration, wherein in the locked configuration the first and second engagement features are configured to engage one another to prevent relative movement of the gripping assembly relative to the handle assembly, wherein in the movable configuration the cam feature is configured to contact the gripping assembly to flex the second engagement feature away from the first engagement feature allowing the gripping assembly to move relative to the handle assembly.

### EXAMPLE 23

An apparatus comprising: (a) guide member, the guide member being operable to guide an instrument into a head of a patient; (b) a handle assembly extending proximally from the guide member, the handle assembly comprising: (i) a first engagement feature, (ii) a first cam surface, and (iii) a second cam surface disposed opposite the first cam surface; and (c) a gripping assembly comprising: (i) a second engagement feature, and (ii) a third cam surface, (iii) a fourth cam surface disposed opposite the third cam surface, wherein the gripping assembly is configured to be selectively transitioned between a locked configuration and a movable configuration, wherein in the locked configuration the first and second engagement features are configured to engage one another to prevent relative movement of the gripping assembly relative to the handle assembly, wherein in the movable configuration the first and second cam surfaces are configured to compress against the third and fourth cam surfaces to flex the second engagement feature away from the first engagement feature allowing for the gripping assembly to translate relative to the handle assembly.

### EXAMPLE 24

The apparatus of Example 23, wherein the guide member includes a guide shaft assembly that defines a lumen configured to receive at least one of a guidewire or a dilation catheter therethrough.

### EXAMPLE 25

The apparatus of any one or more of Examples 23 through 24, wherein the handle assembly includes a first body, wherein the first body includes the first engagement feature and the first and second cam surfaces, wherein the gripping assembly includes a second body, wherein the second body includes the second engagement feature and the third and fourth cam surfaces.

### EXAMPLE 26

A method of using a surgical instrument, wherein the surgical instrument includes a guide member, a handle assembly, and a gripping assembly, wherein the guide member is operable to guide an instrument into a head of a patient, the method comprising: (a) applying a compression force to force a cam surface of one of the handle assembly or the gripping assembly against the other of the handle assembly or the gripping assembly to allow for relative translation of the handle assembly and the gripping assembly; (b) while applying the compression force, translating one of the handle assembly or the gripping assembly relative to the other of the handle assembly; and (c) removing the compression force to prevent relative translation of the handle assembly and the gripping assembly.

### EXAMPLE 27

The method of Example 26, wherein the handle assembly includes a plurality of engagement teeth, wherein gripping assembly includes a plurality of engagement teeth, wherein applying the compression force further comprises applying the compression force to disengage the engagement teeth of the gripping assembly from the engagement teeth of the handle assembly, wherein removing the compression force engages the engagement teeth of the gripping assembly with the engagement teeth of the handle assembly.

### EXAMPLE 28

The method of any one or more of Examples 26 through 27, wherein the handle assembly extends along a longitudinal axis, wherein applying compression force further comprises applying the compression force in a direction perpendicular to the longitudinal axis, wherein translating one of the handle assembly or the gripping assembly relative to the other of the handle assembly or the gripping assembly further comprises translating one of the handle assembly or the gripping assembly relative to the other of the handle assembly or the gripping assembly along the longitudinal axis.

### EXAMPLE 29

A method of using an apparatus, wherein the apparatus includes a guide member, a handle assembly, and a gripping assembly, wherein the guide member is operable to guide an instrument into a head of a patient, wherein the handle assembly includes a first engagement feature, wherein the gripping assembly includes a second engagement feature, the method comprising: (a) applying a compression force to force a cam surface of one of the handle assembly or the gripping assembly against a cam surface of the other of the handle assembly or the gripping assembly so that the first engagement feature of the handle assembly is disengaged from the second engagement feature of the gripping assembly; (b) while applying the compression force, translating one of the handle assembly or the gripping assembly relative to the other of the handle assembly or the gripping assembly; and (c) removing the compression force to engage the first engagement feature of the handle assembly with the second engagement feature of the gripping assembly.

### EXAMPLE 30

The method of Example 29, wherein the engagement feature of the handle assembly includes a plurality of engagement teeth, wherein the engagement feature of the gripping assembly includes a plurality of engagement teeth, wherein applying the compression force further comprises applying the compression force to disengage the engagement teeth of the gripping assembly from the engagement teeth of the handle assembly, wherein removing the compression force disengages the engagement teeth of the gripping assembly from the engagement teeth of the handle assembly.

### EXAMPLE 31

The method of any one or more of Examples 29 through 30, wherein the handle assembly extends along a longitudinal axis, wherein applying compression force further comprises applying compression force in a first direction perpendicular to the longitudinal axis, wherein translating one of handle assembly or the gripping assembly relative to the other of the handle assembly further comprises translating one of the handle assembly or the gripping assembly relative to the other of the handle assembly along the longitudinal axis.

### EXAMPLE 32

The method of Example 31, wherein applying compression force in the first direction causes the second engagement feature to deflect in a second direction, away from the first engagement feature, the second direction being perpendicular to the first direction, the second direction also being perpendicular to the longitudinal axis.

### EXAMPLE 33

The method of any one or more of Examples 29 through 30, wherein applying the compression force causes the second engagement feature to deflect away from the first engagement feature along a path that is transverse to a longitudinal axis defined by the handle assembly.

III. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus comprising:
(a) a guide member operable to guide an instrument into a head of a patient;
(b) a handle assembly extending proximally from the guide member, wherein the handle assembly includes a first engagement feature;
(c) a gripping assembly that includes a second engagement feature, wherein the gripping assembly is configured to selectively transition between a locked configuration and a movable configuration, wherein in the locked configuration the first and second engagement features are configured to engage one another to prevent relative movement of the gripping assembly relative to the handle assembly; and
(d) a grip adjustment assembly that includes a cam feature disposed on at least one of the handle assembly or the gripping assembly, wherein in the movable configuration the cam feature of the grip adjustment assembly is configured to flex the second engagement feature away from the first engagement feature allowing the gripping assembly to move relative to the handle assembly.

2. The apparatus of claim 1, wherein the handle assembly includes an outer surface, wherein the cam feature is disposed on the outer surface.

3. The apparatus of claim 1, wherein in the movable configuration the cam feature is configured to contact the gripping assembly to flex the second engagement feature away from the first engagement feature allowing the gripping assembly to move relative to the handle assembly.

4. The apparatus of claim 1, wherein in the locked configuration the first and second engagement features are configured to engage one another to prevent translation of the gripping assembly relative to the handle assembly, wherein in the movable configuration the cam feature is configured to contact the gripping assembly to flex the second engagement feature away from the first engagement feature allowing the gripping assembly to translate relative to the handle assembly.

5. The apparatus of claim 1, wherein one of the handle assembly or the gripping assembly includes an outwardly projecting surface, wherein the cam feature is disposed on outwardly projecting surface.

6. The apparatus of claim 1, wherein the cam feature includes first and second cam surfaces disposed on opposing first and second outwardly projecting surfaces of the handle assembly.

7. The apparatus of claim 6, wherein of the gripping assembly includes first and second arms that are configured to contact the first and second cam surfaces in the movable configuration.

8. The apparatus of claim 7, wherein the first and second arms are configured to be separated a distance from the first and second cam surfaces in the locked configuration.

9. The apparatus of claim 7, wherein the first and second arms include first and second inwardly facing projections, wherein the first and second inwardly facing projections are configured to contact the first and second cam surfaces in the movable configuration.

10. The apparatus of claim 7, wherein the first and second arms are configured to flex outwardly due to force exerted by the first and second cam surfaces in the movable configuration.

11. The apparatus of claim 7, wherein the first engagement feature includes a first plurality of engagement teeth, wherein the second engagement feature includes a second plurality of engagement teeth disposed on the first arm, wherein the plurality of engagement teeth are configured to engage the first plurality of engagement teeth in the locked configuration.

12. The apparatus of claim 1, wherein the first engagement feature includes a first plurality of engagement teeth, wherein the second engagement feature includes a second plurality of engagement teeth, wherein in the locked configuration at least one tooth of the first plurality of engagement teeth is configured to be in locking engagement with least one tooth of the first plurality of engagement teeth.

13. The apparatus of claim 1, wherein the gripping assembly includes a plurality of gripping handles.

14. The apparatus of claim 1, wherein the handle assembly includes a first body, wherein the first body includes the first engagement feature and the cam feature, wherein the gripping assembly includes a second body, wherein the second body includes the second engagement feature.

15. The apparatus of claim 1, wherein the gripping assembly is formed of an elastomeric material.

16. The apparatus of claim 1, further comprising a dilation catheter, the guide member being operable to guide the dilation catheter into a head of a patient, wherein the dilation catheter is slidable relative to the guide member, wherein the dilation catheter comprises an expandable dilator.

17. The apparatus of claim 16, further comprising a guidewire, wherein the guidewire is slidably disposed in the dilation catheter.

18. The apparatus of claim 1, further comprising a shaft rotation assembly, wherein the shaft rotation assembly is operable to rotate the guide member about the longitudinal axis.

19. An apparatus comprising:
(a) a guide member, the guide member being operable to guide an instrument into a head of a patient;
(b) a handle assembly extending proximally from the guide member, the handle assembly comprising:
(i) a first engagement feature, and
(ii) a cam feature disposed on an outer surface of the handle assembly; and
(c) a gripping assembly that includes a second engagement feature, wherein gripping assembly is configured to selectively transition between a locked configuration and a movable configuration, wherein in the locked configuration the first and second engagement features are configured to engage one another to prevent relative movement of the gripping assembly relative to the handle assembly, wherein in the movable configuration the cam feature is configured to contact the gripping assembly to flex the second engagement feature away from the first engagement feature allowing the gripping assembly to move relative to the handle assembly.

20. An apparatus comprising:
(a) a guide member operable to guide an instrument into a head of a patient;
(b) a handle assembly extending proximally from the guide member, the handle assembly comprising:
(i) a first engagement feature,
(ii) a first cam surface, and
(iii) a second cam surface disposed opposite the first cam surface; and
(c) a gripping assembly comprising:
(i) a second engagement feature, and
(ii) a third cam surface,
(iii) a fourth cam surface disposed opposite the third cam surface,
wherein the gripping assembly is configured to be selectively transitioned between a locked configuration and a movable configuration, wherein in the locked configuration the first and second engagement features are configured to engage one another to prevent relative movement of the gripping assembly relative to the handle assembly, wherein in the movable configuration the first and second cam surfaces are configured to compress against the third and fourth cam surfaces to flex the second engagement feature away from the first engagement feature allowing for the gripping assembly to translate relative to the handle assembly.
